(19) United States
(12) Patent Application Publication
(10) Pub. No.: US 2026/0272723 A1
(43) Pub. Date: Sep. 17, 2026

(54) EXPANDING WOUND DRESSING

(71) Applicant: RevMedx, Inc., Wilsonville, OR (US)

(72) Inventors: John Steinbaugh, Wilsonville, OR (US); Andrew Barofsky, Lake Oswego, OR (US)

(21) Appl. No.: 19/565,400

(22) Filed: Mar. 12, 2026

Related U.S. Application Data
(60) Provisional application No. 63/770,535, filed on Mar. 12, 2025.

(51) Int. Cl. A61F 13/15; A61F 13/00; A61F 13/53

(52) U.S. Cl. CPC A61F 13/15203; A61F 13/00063; A61F 13/53; A61F 2013/1539; A61F 2013/530802

(57) ABSTRACT

Embodiments provide self-expanding wound dressings that include an outer layer having a first liquid permeability; an inner pouch positioned within the outer layer, the inner pouch having a second liquid permeability; and a liquid-expandable layer positioned within the inner pouch, wherein the liquid-expandable layer comprises a plurality of liquid-expandable articles, wherein the first liquid permeability is lower than the second liquid permeability. Also provided are methods of treating an individual having a bleeding wound, the methods including applying a self-expanding wound dressing to the wound, as well as methods of manufacturing a wound dressing, the methods including forming an absorbent material into a plurality of liquid-expandable articles, positioning the liquid-expandable layer in an inner pouch, and positioning the inner pouch within an outer layer to form the wound dressing.

### CROSS-REFERENCE TO RELATED APPLICATION

This application claims the priority benefit of the earlier filing date of U.S. Provisional Application No. 63/770,535, filed Mar. 12, 2025, which is hereby incorporated herein by reference in its entirety.

### TECHNICAL FIELD

Embodiments herein relate to methods, compositions, and devices for controlling bleeding and treating wounds.

### BACKGROUND

Uncontrolled external hemorrhage is the leading cause of death on the battlefield and the second leading cause of death in civilian trauma. At the point of injury, every drop of blood is precious and cannot be replaced until the casualty reaches the next echelon of care. Wounds sustained from IED's, blasts, and small caliber missiles can cause open and penetrating wounds that are difficult to pack with gauze-based dressings. Application of sufficient pressure on bleeding vessels to control hemorrhage is a constant challenge in austere environments. Further, treatment of a large wound can quickly deplete a medic's available supply of dressings.

A principal method for treating bleeding wounds is to stop the flow of blood by applying pressure with a bandage to facilitate formation of a clot. However, current wound dressings are often too stiff and too rigid to fit into a narrow space of a cavity wound or, if sufficiently pliable, do not adequately conform to irregular tissues geometries to cause rapid and effective hemostasis. Bandage and gauze technologies are hard to pack and hold into these wounds and generally fail to adequately stop blood loss.

Hemorrhage control in prolonged field care presents unique challenges that drive the need for enhanced point of injury treatment capabilities to maintain patient stability beyond the Golden Hour. Anticipated peer to peer conflicts where air superiority is unlikely and robust evacuation capability is less available mandates the US Military need for new advanced hemorrhage control capabilities that can be applied beyond doctrinal planning timelines until the patient can be delivered to definitive care. Prolonged exposure to unchallenged bioburden may disrupt hemostasis and increases the likelihood of infection. Virulent activity of organisms, such as Staphylococcus aureus and group A Streptococci, have evolved mechanisms to modulate blood coagulation. Traumatic injury also poses a significant risk of intravascular infections such as sepsis and infective endocarditis. Infection risks increase with concomitant burn injuries that are common with complex blast and penetrating trauma.

Accordingly, there remains a need for a more effective way to treat hemorrhagic injuries.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1A illustrates an exemplary self-expanding wound dressing prior to expansion.

FIG. 1B depicts the self-expanding wound dressing of FIG. 1A following expansion of the liquid expandable layer, in accordance with various embodiments.

FIG. 1C provides a cutaway view of an exemplary self-expanding wound dressing following expansion.

FIG. 1D provides a cross-sectional, schematic view of an exemplary self-expanding wound dressing prior to expansion.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide self-expanding wound dressings comprising a liquid-expandable layer. In general, embodiments include methods for treating hemorrhagic injuries. More specifically, there is provided a method to effect rapid hemostatic response and control hemorrhage by introducing a self-expanding wound dressing into, on, or around a bleeding cavity. An embodiment also provides a method of preparing or manufacturing such a self-expanding wound dressing.

In the following description, unless further particularized or otherwise noted, the term “liquid-expandable” is intended to refer to any material or substance that expands in occupied volume upon contact with a liquid.

Embodiments provided herein are directed to self-expanding wound dressings that include a plurality of containment layers, and a liquid-expandable layer contained within the plurality of containment layers. In an embodiment, an outer layer may form an outer pouch constructed from multiple pieces of material, or the outer layer may be constructed from a single piece of material. An inner pouch, such as a porous pouch, for example a fibrous mesh pouch, may be positioined interior to the outer layer to provide a multi-layered structure. In embodiments, the layers can be constructed in various ways to form a fully sealed or partially open structure. The outer layer and inner pouch are configured to control the rate at which liquid, such as blood, reaches the liquid-expandable layer.

In various embodiments, the liquid-expandable layer may include a plurality of liquid-expandable articles, such as described in U.S. Pat. No. 8,828,050, hereby incorporated by reference herein. In various embodiments, the plurality of liquid-expandable articles expand to form expanded articles upon contact with a liquid. In certain embodiments, the liquid-expandable layer may be formed into a sheet, such as described in U.S. Pat. No. 11,020,279, hereby incorporated by reference herein.

In some embodiments, the self-expanding wound dressings may have a peripheral boundary of predetermined size and shape sufficient to cover or fit into an afflicted wound area. While it is noted that the self-expanding wound dressings may have a specific size and shape, they are also intended to be sufficiently pliable to be compressed or otherwise manipulated to place them into or on wounds of various shapes, configurations, and sizes.

In various embodiments, the outer layer has a first liquid permeability, and the inner pouch has a second liquid permeability, the second liquid permeability being greater than the first liquid permeability. In this manner, as liquid penetrates the outer layer, the liquid will more readily pass through the material of the inner pouch to the interior thereof to contact the liquid-expandable layer.

As used herein, liquid permeability may be determined by the rate at which a given volume of fluid passes through the layer/pouch or by the volume of fluid that passes through the layer/pouch in a given amount of time.

In various embodiments, liquid may pass through the layer/pouch through one or more transport mechanisms or material properties including permeability, porosity, capillary transport, wetting behavior, fluid distribution, hydraulic resistance, diffusion characteristics, or combinations thereof.

The differential noted above with respect to liquid permeability provides a controllable rate of liquid ingress to the liquid-expandable layer through the outer layer and inner pouch. As such, the outer layer and inner pouch may be configured to limit the amount of liquid or the rate of the uptake of the liquid to the liquid-expandable layer at the interior of the wound dressing.

In various embodiments, the permeability of the outer layer may be selected to reduce the rate at which liquid, such as blood, would otherwise penetrate the wound dressing and reach the liquid-expandable layer. By decreasing the permeability or flow of liquid through the outer layer relative to the inner pouch, the ingress of liquid to the liquid-expandable articles may be moderated, thereby slowing the rate at which the liquid-expandable articles absorb fluid and expand. This controlled delay in expansion may improve usability of the wound dressing during application. For example, by slowing the onset of expansion, a user may be afforded additional time to position the dressing within a wound cavity, adjust placement, or complete packing of the wound prior to the dressing exerting significant expansion forces. In this manner, the reduced permeability of the wound dressing provided by the outer layer may help prevent premature expansion that could otherwise make insertion, repositioning, or adjustment of the wound dressing more difficult once pressure begins to develop within the wound site.

In certain embodiments, even relatively small differences in liquid permeability or flow rate between the outer layer and inner pouch may provide meaningful improvements in usability. For instance, a delay of only a few seconds in the onset or rate of expansion, compared to the rate without the multi-layered structure, may allow a user additional time to correctly place the dressing, reposition it within the wound cavity, or complete packing steps before expansion pressure increases. These brief delays may improve placement accuracy, reduce the likelihood of premature expansion interfering with insertion, and ultimately enhance overall wound management performance of the dressing.

As such, in certain embodiments, an outer layer of a self-expanding wound dressing, as described herein, may add 2-20 seconds of additional time before liquid reaches the liquid-expandable layer, compared to a self-expanding wound dressing with only an inner pouch.

Also provided is a method of treating an individual having a bleeding wound, the method including applying a self-expanding wound dressing as disclosed herein to the wound. Additional embodiments are methods of manufacturing a wound dressing, the methods including forming an absorbent material into a plurality of liquid-expandable articles, positioning the plurality of liquid expandable articles inside an inner pouch, and positioning the inner pouch inside an outer layer to form the wound dressing.

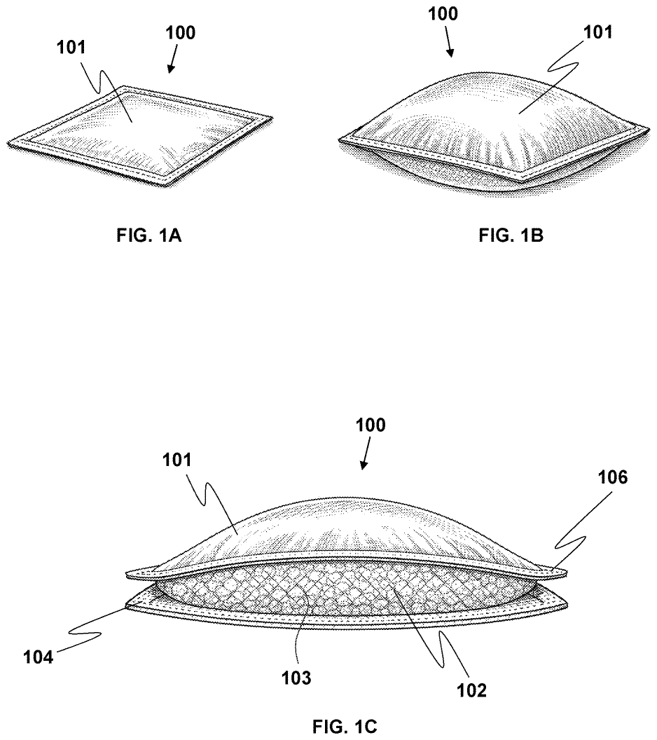

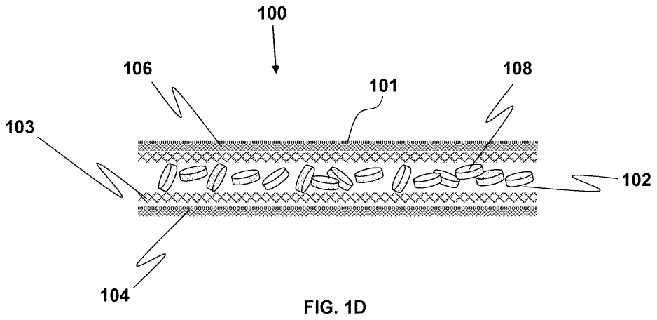

FIG. 1A illustrates an exemplary self-expanding wound dressing prior to expansion, and FIG. 1B depicts the self-expanding wound dressing of FIG. 1A following expansion of the liquid-expandable layer, in accordance with various embodiments. FIG. 1C provides a cutaway view of an exemplary self-expanding wound dressing following expansion. FIG. 1D provides a cross-sectional view of an exemplary self-expanding wound dressing prior to expansion.

As shown in FIGS. 1C and 1D, self-expanding wound dressing 100 includes a liquid-expandable layer 102 positioned within an inner pouch 103, which is further positioned within outer layer 101. Outer layer 101 is shown constructed from a bottom layer 104 and a top layer 106, although as noted above outer layer 101 can be constructed from 1 or more pieces of material, as desired.

In some embodiments, bottom layer 104 and a top layer 106 may be joined at their peripheries to form one or more cells or pockets to receive liquid-expandable layer 102 therein. In particular embodiments, these cells or pockets may form a pattern or array. In such embodiments, bottom layer 104 and a top layer 106 may be joined at additional points or lines internal to their periphery. In some embodiments, the cells or pockets may be formed by welding together bottom layer 104 and a top layer 106. In other embodiments, the cells or pockets may be formed by stitching together bottom layer 104 and a top layer 106. In other embodiments, the cells or pockets may be formed by heat bonding together bottom layer 104 and a top layer 106. In other embodiments, the cells or pockets may be formed by gluing together bottom layer 104 and a top layer 106.

In various embodiments, liquid expandable layer 102 includes a plurality of liquid-expandable articles 108 that are positioned inside inner pouch 103.

As noted above, in certain embodiments, outer layer 101 may be formed from a single, continuous material rather than separate sheets. For example, outer layer 101 may be formed from a continuous tube or sleeve of material that surrounds inner pouch 103. In such embodiments, the tubular material may be sealed, stitched, welded, glued, or otherwise joined at one or more ends to enclose inner pouch 103 within the interior of the tube.

As shown in FIGS. 1C and 1D, outer layer 101 may be flexible and porous. Exemplary porous materials for outer layer 101 may include fabrics, meshes, and/or gauzes. For example, outer layer 101 may be made of woven fabrics, nonwoven fabrics, melt-blown webs, foams, spun-bonded webs, thermal-bonded webs, spun-laced webs, paper, and/or thermally-embossed nonwoven fabrics. More precisely, specific, non-limiting examples of suitable first and second outer layers include woven fabrics, knitted fabrics, or non-woven fabrics of an organic polymer such as cotton, polyvinyl alcohol, or cellulose; paper; and perforated films of polyvinyl alcohol. In various embodiments, outer layer 101 may be elastic or non-elastic. In some embodiments, outer layer 101 may be non-porous.

In various embodiments defining a multi-piece outer layer, bottom layer 104 and top layer 106 may be made of the same material. In other embodiments, bottom layer 104 and top layer 106 may be made of different materials. For example, bottom layer 104 may be porous and top layer 106 may be non-porous. In various embodiments, bottom layer 104 may exhibit different fluid permeability properties than top layer 106. In certain embodiments, one or both of bottom layer 104 and top layer 106 may provide resistance to liquid ingress, thereby controlling the rate at which liquid reaches liquid-expandable layer 102. In various embodiments, one or both of bottom layer 104 and top layer 106 may exhibit a lower degree of liquid permeability than the inner pouch 103. For such embodiments, the permeability of one or both of bottom layer 104 and top layer 106 may be reduced relative to the permeability of the inner pouch 103.

In embodiments, outer layer 101 may exhibit a liquid permeability that is approximately 1-25% lower than the permeability of the inner pouch 103. In an embodiment, outer layer 101 may exhibit a liquid permeability that is approximately 1-10% lower than the permeability of the inner pouch 103. In other embodiments, the permeability of outer layer 101 may be approximately 10-20% lower than the permeability of the inner pouch 103. In still other embodiments, the permeability of outer layer 101 may be more than 20% lower than the permeability of the inner pouch 103. Such differences in permeability may contribute to controlling the rate at which liquid contacts liquid expandable layer 102.

In various embodiments, outer layer 101 may be configured to reduce the rate of liquid flow into the interior of the wound dressing relative to the inner pouch 103. By controlling the rate at which liquid, such as blood, passes through the outer layer, the flow of liquid to the liquid-expandable layer may be moderated. In certain embodiments, the outer layer 101 may exhibit a liquid flow rate that is lower than the liquid flow rate through the inner pouch 103. For example, the liquid flow rate through the outer layer may be approximately 1-10% lower than the liquid flow rate through the inner pouch 103. In other embodiments, the liquid flow rate through the outer layer 101 may be approximately 10-20% lower than the liquid flow rate through the inner pouch 103. In still other embodiments, the liquid flow rate through the outer layer may be more than 20% lower than the liquid flow rate through the inner pouch 103. In this manner, the outer layer 101 may function to regulate the rate at which liquid reaches the interior of the dressing, thereby influencing the timing and rate at which liquid-expandable layer 102 expands.

In various embodiments, the outer layer 101 may be configured to delay the initiation of expansion of liquid-expandable layer 102 by controlling the rate at which liquid reaches the interior of the wound dressing. By moderating the rate at which liquid, such as blood, passes through the outer layer 101 and reaches inner pouch 103 and the liquid-expandable articles 108 contained therein, the onset of expansion of liquid-expandable layer 102 may be delayed for a controlled period of time following initial contact by the wound dressing with liquid. In certain embodiments, the presence and material characteristics of outer layer 101 may delay the initiation of expansion by approximately 1-3 seconds relative to a configuration lacking an outer layer. In other embodiments, the delay in initiation of expansion may be approximately 3-5 seconds. In still other embodiments, the delay may be approximately 5-10 seconds. In further embodiments, the delay in initiation of expansion may be greater than 10 seconds.

In various embodiments, the presence of outer layer 101 may reduce the rate at which liquid reaches the liquid-expandable layer even when the permeability or liquid flow rate of the outer layer is similar to, or not lower than, that of the inner pouch 103. For example, the addition of one or more outer layers may increase the overall path length that liquid must travel before reaching the liquid-expandable layer after passing through the inner pouch 103. In certain embodiments, the liquid must first pass through outer layer 101, then through porous pouch 101, to contact the liquid-expandable articles. This multi-layer pathway may create additional flow resistance or fluid distribution effects that slow the rate at which liquid reaches the liquid-expandable layer. As a result, even when outer layer 101 has permeability or liquid flow characteristics comparable to the inner pouch 103, the overall structure of the wound dressing may still reduce the effective rate of liquid ingress to the liquid-expandable layer. This configuration may provide a short but useful delay in expansion, which may assist a user in positioning or adjusting the wound dressing prior to the onset of expansion forces.

In various embodiments, outer layer 101 may also improve the tactile qualities of the wound dressing during handling and placement. For example, the material selected for the outer layer may provide a softer, smoother, or otherwise more favorable feel when the dressing is handled by a user. Such tactile characteristics may improve the ease with which the wound dressing can be grasped, manipulated, and positioned within a wound site during application. In certain embodiments, the outer layers may be formed from fabrics, meshes, gauzes, or other fibrous materials that provide a compliant and comfortable surface texture that may be easier for a user to handle compared to the materials of the liquid-expandable layer or the inner pouch positioned within the dressing.

In some embodiments, the tactile properties of outer layer 101 may also facilitate improved control during placement of the wound dressing within a wound cavity. For example, the outer layer may provide a degree of friction, flexibility, or conformability that allows a user to more easily guide the dressing into irregular wound spaces or cavities. The outer layer may also provide structural cohesion that helps maintain the shape and integrity of the dressing during insertion and positioning. In this manner, the outer layer may contribute not only to fluid management and expansion control, but also to improved usability and handling characteristics during deployment of the wound dressing. In certain embodiments, outer layer 101 may further provide a protective interface between the internal components of the wound dressing and surrounding tissue. For example, outer layer 101 may shield the liquid-expandable articles 108 and inner pouch 103 from direct contact with tissue during placement, which may improve the overall feel of the dressing during insertion and reduce the likelihood that internal components create localized pressure points or irregular tactile sensations during application. As a result, the presence of outer layer 101 may enhance the overall tactile performance of the wound dressing while also supporting the functional objectives of the device.

FIG. 1B illustrates the expanded self-expanding wound dressing 100 of FIG. 1A following contact with a liquid. As shown in FIGS. 1A and 1B, liquid-expandable layer 102 (not shown) expands into an expanded layer (as shown in FIG. 1B by the increase in volume of dressing 100) upon contact with a liquid. In various embodiments, the liquid may be an aqueous solution, such as a bodily fluid. For example, in some embodiments, the liquid may be blood or serum. In various embodiments, once expanded, expanded layer may be soft and pliable. Without being limited to any particular theory, this quality may permit self-expanding wound dressing 100 to conform to irregular wound crevices, gaps, and fissures.

In various embodiments, the expanded layer occupies a volume greater than that of liquid-expandable layer 102. In various embodiments, the average volume ratio of the expanded layer to the liquid-expandable layer is at least 2× (in other words, the volume of the expanded layer is capable of expanding to at least 2 times the volume of the liquid-expandable layer). In other embodiments, the average volume ratio of the expanded layer to the liquid-expandable layer is at least 4×. In still other embodiments, the average volume ratio of the expanded layer to the liquid-expandable layer is at least 8×. In yet other embodiments, the average volume ratio of the expanded layer to the liquid-expandable layers is at least 10×.

In various embodiments, liquid-expandable layer 102 may be capable of expanding to 80% or greater of its maximum expansion capacity in 60 seconds or less following immersion in water or saline. In other embodiments, liquid-expandable layer 102 may be capable of expanding to 80% or greater of its maximum expansion capacity in 30 seconds or less following immersion in water or saline. In still other embodiments, liquid-expandable layer 102 may be capable of expanding to 80% or greater of its maximum expansion capacity in 10 seconds or less following immersion in water or saline. In yet other embodiments, liquid-expandable layer 102 may be capable of expanding to 80% or greater of its maximum expansion capacity in 5 seconds or less following immersion in water or saline. In certain environments, in use, liquid-expandable layer 102 may be unable to reach its maximum expansion capacity as it may be constrained in some manner, such as by the dimensions of the wound.

In various embodiments, the liquid expandable layer 102 may be uniformly distributed in self-expanding wound dressing 100. In other embodiments, the liquid expandable layer 102 may be asymmetrically distributed in self-expanding wound dressing 100. In this way, segments of self-expanding wound dressing 100 may exhibit an ability to self-expand upon contact with a liquid, while other segments of the dressing may not.

In various embodiments, liquid expandable layer 102 may include an absorbent material including, but not limited to, a sponge or fibrous material. In various embodiments, the absorbent material may include a polysaccharide such as, but not limited to, cellulose, starch, chitin, or chitosan. In some embodiments, liquid expandable layer 102 may include regenerated cellulose sponge. In other embodiments, the absorbent material may include synthetic sponges such as, but not limited to, various polyvinyl alcohol (PVA) polymers and derivatives thereof having desirable physical and mechanical properties. In various embodiments, liquid expandable layer 102 may be biodegradable and/or bioabsorbable.

In various embodiments, the absorbent material may form a contiguous sheet. In other embodiments, the absorbent material may include a plurality of distinct articles that move relatively independent of one another.

In various embodiments, liquid expandable layer 102 may include a compressed material. As used herein, the term “compressed material” refers to a material that has a first size/dimension or form factor in a non-compressed state, and has a second, reduced size/dimension or form factor in a compressed state. In various embodiments, the compressed material may remain in essentially a compressed state indefinitely until hydrated. For these embodiments, and without being limited to any particular theory, the compressed material, when hydrated, may rapidly expand in an effort to assume its pre-compression dimensions. In this way, the compressed material may store additional mechanical energy in a compressed state, as compared to the non-compressed state, that is released when exposed to a liquid, thus causing liquid expandable layer 102 to quickly expand. In various embodiments, the absorbent material may be compressed by heat compression or any other suitable compression method known in the art.

In various embodiments, self-expanding wound dressing 100 may be sufficiently flexible to insert and pack into wounds.

In various embodiments, liquid expandable layer 102 includes liquid-expandable articles 108. For such embodiments, liquid-expandable articles 108 may include one or more absorbent materials, which may be compressed materials in certain embodiments. In some embodiments, liquid-expandable articles 108 may include regenerated cellulose sponge. The liquid-expandable articles 108 are adapted to expand into expanded articles upon contact with a liquid. In various embodiments, the expanded articles may have a volume greater than the liquid-expandable articles 108. As described above with respect to FIGS. 1A and 1B, in various embodiments, the average volume ratio of expanded articles to liquid-expandable articles may be between about 2× and about 12×, or even more. Additionally, in various embodiments, liquid-expandable articles 108 may be capable of expanding to 80% or greater of their maximum expansion capacity in 60 seconds or less following immersion in water or saline, such as about 30 second, about 10 seconds, about 5 seconds, or even less.

In various embodiments, self-expanding wound dressing 100 may include at least 3 liquid-expandable articles 108, such as about 10, 50, 100, or even more liquid-expandable articles. In various embodiments, the occupied volume of each liquid-expandable article 108 prior to expansion may be from 0.7 mm³ to 7000 mm³, such as from about 500 mm³ to 5000 mm³. In some embodiments, the volume of each liquid-expandable article may be greater than 1 mm³ prior to expansion, such as about 5 mm³, about 10 mm³, about 50 mm³, or about 100 mm³, or even greater. In various embodiments, liquid-expandable articles 108 may all be uniform in size or may include a mixture of sizes.

In various embodiments, liquid-expandable articles 108 may have one or more predetermined shapes, based on adjustments to the length, width, diameter, or cross-sectional shape. Without being limited by theory, the shape, size and/or pattern of liquid-expandable articles 108 may influence the pliability of self-expanding wound dressing 100, as well as the ability of the self-expanding wound dressing to fit into, expand, fill, partially fill and conform to a wound cavity. In addition, the shape may assist expanded articles in retaining a desired position in the wound cavity. The predetermined shape of liquid-expandable articles 108 may include articles with round, triangular, square, rectangular, hexagonal, conical, or octagonal cross-sections. In various embodiments, predetermined shapes having multiple projections (e.g., a star) may be used. In other embodiments, self-expanding wound dressing 100 may include liquid-expandable articles 108 with haphazard, random, irregular, or jagged shapes. In various embodiments, self-expanding wound dressing 100 may include liquid-expandable articles 108 of two or more predetermined shapes. In other embodiments, self-expanding wound dressing 100 may include liquid-expandable articles 108 that have a mixture of predetermined shapes and/or irregular shapes.

In various embodiments, the predetermined shape of liquid-expandable articles 108 may define any shape having first major outer surface and a second major outer surface. For such embodiments, the first major outer surface and the second major outer surface may be substantially parallel to one another. In various embodiments, the average distance between the outer surfaces may be from 0.5 mm to 20 mm, such from 1 mm to 10 mm, or from 1 mm to 5 mm. In various embodiments, such as the illustrated embodiment, liquid-expandable articles 108 may be substantially in the form of a disk or cylinder. For such embodiments, the average diameter of the first major outer surface and the second major outer surface may be from 1 mm to 20 mm, such from 5 mm to 10 mm. In various embodiments, self-expanding wound dressing 200 may include liquid-expandable articles 108 having the same average diameter or a mixture of liquid-expandable articles 108 having different average diameters.

In various embodiments, the predetermined pattern may define one or more regions without any liquid-expandable articles. These article-free regions may be used as fold lines that allow the self-expanding wound dressing to be folded at a particular location on the dressing. Some embodiments may include one or more fold lines, which may increase the flexibility of the self-expanding wound dressing, or which may help the self-expanding wound dressing conform to a particular would size or shape or fit into a desired packaging. In additional embodiments, one or more article-free regions may be used as cut lines that function to identify where a user may cut the dressing without being impeded by liquid expandable articles.

In various embodiments, self-expanding wound dressings described herein may further include one or more therapeutic agents. For example, in some embodiments, the self-expanding wound dressing may be impregnated, suffused, or coated with one or more therapeutic agents, and/or the one or more therapeutic agents may be dispersed throughout the self-expanding wound dressing. In some embodiments, the one or more therapeutic agents may be provided only on the liquid-expandable articles, only on an outer layer or layers, or a combination of both. In various embodiments, the one or more therapeutic agents may be selected from the group consisting of analgesics, steroids, antihistamines, anesthetics, bactericides, disinfectants, fungicides, vasoconstrictors, chemotherapeutic drugs, antibiotics, keratolytics, cauterizing agents, antiviral drugs, epidermal growth factor, fibroblast growth factors, transforming growth factors, glycoproteins, fibrinogen, fibrin, humectants, preservatives, lymphokines, cytokines, odor controlling materials, vitamins, and clotting factors.

In various embodiments, the one or more therapeutic agents may include hemostatic agent(s). For example, the one or more therapeutic agents may include chitosan or a derivative of chitosan. In other embodiments, the one or more therapeutic agents may include kaolin. In other embodiments, the one or more therapeutic agents may be selected from the group consisting of diatomaceous earth, silica, clays, minerals, attapulgite, bentonite, zeolite, and bioactive glasses.

In various embodiments, the one or more therapeutic agents may include an inorganic salt. Examples of an inorganic salt include, but are not limited to, a divalent ion selected from the group consisting of zinc, copper, magnesium, calcium and nickel, as well as CaO, CaCl₂, AgNO₃, Ca(NO₃)₂, Mg(NO₃)₂, Zn(NO₃)₂, NH₄NO₃, AgCl, Ag₂O, zinc acetate, magnesium acetate, calcium citrate, zinc citrate, magnesium citrate, magnesium chloride, magnesium bromide, zinc chloride, zinc bromide, calcium bromide, calcium acetate and calcium phosphate.

In various embodiment, the one or more therapeutic agents may include an antimicrobial metal. For such embodiments, the antimicrobial metal may include an antimicrobial metal metallic copper (Cu), cuprous oxide (CU2O), cupric oxide (CuO), or cuprous iodide (CuI), or combinations thereof. In various embodiments, the antimicrobial metal may include silver or copper

In certain embodiments, outer layer 101 may also function as delivery substrates for one or more therapeutic agents. For example, outer layer 101 may be impregnated, coated, treated, or otherwise combined with one or more therapeutic agents such that the therapeutic agents are positioned on or within the materials forming the outer layer. In this configuration, the therapeutic agents may be delivered directly to the wound interface as the wound dressing is placed within or against the wound site. Because outer layer 101 may form the exterior surfaces of the wound dressing that contact surrounding tissue and fluids, the outer layer may provide an effective platform for distributing therapeutic agents across the wound area. In certain embodiments, delivering the therapeutic agents through the outer layer may provide improved exposure of the wound site to the therapeutic agents compared to placement solely within the internal components of the dressing, such as the liquid-expandable articles 108 or inner pouch 103.

In various embodiments, the outer layer may be particularly suitable for delivering therapeutic agents that benefit from direct contact with wound tissue or wound exudate. For example, hemostatic agents, antimicrobial agents, analgesics, or growth factors may be incorporated into the outer layer 101 so that these agents are available at the wound interface immediately upon placement of the dressing. In certain embodiments, the outer layer may provide a relatively large surface area for carrying or releasing such therapeutic agents, which may facilitate distribution of the agents across the wound site. In this manner, outer layer 101 may serve not only structural and fluid-control functions, but may also act as a functional delivery layer that supports therapeutic treatment of the wound.

In an embodiment, suitable materials for the outer layer(s) are described in US 2021/0368890 and US 2023/0263931, which are hereby incorporated by reference herein.

In various embodiments, the self-expanding wound dressing may include one or more markers for identifying the location of the dressing in a wound and/or facilitating removal of the dressing from the wound. In such embodiments, the marker may be coupled to the first and/or second outer layers. In other embodiments, the marker may be coupled to the liquid expandable articles. In some embodiments, the marker may include a radio-frequency identification (RFID) tag. In other embodiments, the marker may include a radiopaque material. For example, the marker may include a radiopaque filament, bead, ball, sphere, wire, or strip. In other examples, the marker may include a barium sulfate-infused polymer, such as polypropylene. In still other examples, at least a portion of the self-expanding wound dressing may be suffused with a radiopaque material. In yet another example, at least a portion of the self-expanding wound dressing may be coated with a radiopaque material.

Also provided herein are methods for effecting rapid hemostatic response and hemorrhage control by applying a self-expanding wound dressing to a bleeding wound. For example, a self-expanding wound dressing may be delivered into a wound cavity where the liquid-expandable layer may contact blood within the cavity and subsequently expand to form an expanded layer.

In various embodiments, applying a self-expanding wound dressing to a wound may include applying the self-expanding wound dressing by hand. In some embodiments, the self-expanding wound dressing may be applied to a wound using a securing adhesive. In another embodiment, the self-expanding wound dressing may be wrapped around a wound site, such as around an arm, leg, torso, etc.

In various embodiments, applying a self-expanding wound dressing to a wound may include applying the self-expanding wound dressing using an applicator. For example, the applicator may include a body, a tip, and a delivery mechanism. The body defines a storage chamber for the self-expanding wound dressing and has an opening for deploying the self-expanding wound dressing from the storage chamber. A tip may cover the opening and has a closed state wherein the tip maintains the self-expanding wound dressing in the storage chamber and an open state wherein the self-expanding wound dressing can be deployed through the opening and the tip.

Exemplary wounds often arise from, but are not limited to, traumatic accidents, projectiles from weapons, or improvised explosive devices that may create large soft tissue wounds in the calf, thigh, buttocks or shoulder that result in severe bleeding. Such wounds may be associated with an arterial puncture, a venous puncture, an arterial laceration and/or a venous laceration. Each wound can have a unique size and/or shape. Often, the extent of the tissue damage cannot be determined until emergent care can be provided. The use of the self-expanding wound dressings disclosed herein may allow for the treatment of several different wound types.

A method of preparing a self-expanding wound dressing in accordance with various embodiments is also provided. In some embodiments, a liquid-expandable material may be prepared by compressing an absorbent material to form a compressed absorbent material. This may be accomplished, for example, using conventional mechanical compression techniques well known to those skilled in the art. In other embodiments, compressing the absorbent material into a liquid-expandable material may include freeze-drying the absorbent material. Exemplary absorbent materials include sponges. In some embodiments, the liquid expandable layer may be adapted to be flexible in accordance with various methods described above.

In other embodiments, the liquid expandable layer may be adapted to be flexible by forming the compressed absorbent material into a plurality of liquid-expandable articles. This may include forming the compressed absorbent material into desirable shapes and sizes. For such embodiments, the liquid-expandable material may be cut using, for example, a die and press. The absorbent material may also be molded directly into desired shapes and sizes. In various embodiments, the absorbent material may be formed into a plurality of liquid-expandable articles by extrusion, pelletization, briquetting, tabletting, or other methods familiar to those skilled in the art. Alternatively, the absorbent material may be mechanically crushed into irregular shaped lumps, with desirable size ranges to be separated out by a classifier.

In various embodiments, the self-expanding wound dressing may be combined with one or more therapeutic agents by impregnating, suffusing, coating or dispersing the one or more therapeutic agents on or throughout the absorbent material. In an embodiment, the therapeutic agent may be sprayed onto the absorbent material. In another embodiment, the absorbent material may be soaked in a therapeutic agent solution.

Embodiments also provide a composition that includes a self-expanding wound dressing with a liquid-expandable layer that is configured to induce hemostasis when contacted with blood and that may be applied to many types of wounds. In various embodiments, the liquid-expandable layer may possess an ability, upon contact with blood, to rapidly expand to form a pliable, shapeable and conformable mass. Without limiting the scope of the disclosure, this mass may exert mechanical pressure on the surface of the wound, as well as interact with blood components to ultimately facilitate the formation of a fluid arresting coagulum within the wound cavity. In various embodiments, the combination of mechanical pressure and enhanced clotting may make the composition able to curtail bleeding without the application of external compression. In other embodiments, liquid-expandable layer may be capable of expanding through a swelling mechanism. While it is noted above that external compression is not necessary, in some embodiments, external compression may be used in conjunction with the self-expanding wound dressing. Such external compression may be provided by an elastic backing material or by a separate elastic or compression material/device.

Without limiting the scope of the disclosure, the devices and compositions disclosed herein may be advantageous for several reasons. In various embodiments, the liquid-expandable dressings disclosed herein have the ability to quickly expand into an expanded dressing. This allows the expanded dressing to quickly fill the wound cavity and provide a rapid hemostatic effect. Additional advantages include improved positioning within the wound, improved tissue apposition, and better conformation to intricate wound contours. The soft, pliable nature of the dressing, in connection with spring-like characteristics, permits the expanded dressing to provide a gentle outward pressure within the wound cavity, without the need to apply excessive pressure that can compromise perfusion to local tissues. Because the expanded articles conform to the wound cavity, pressure is exerted multi-directionally to address all bleeding points. The ability to exert outward pressure against and closely conforming to surrounding tissue surfaces helps the expanded dressing to maintain positioning within the wound cavity in the face of high flow arterial bleeding and deformation during transport of the injured person, maximize the contact and application of material at the sources of bleeding, and ensure constant and gentle, yet effective, compression within the wound cavity (without creating harmful pressure points).

The following provides an example preparation of a self-expanding wound dressing with compressed cellulose sponges, in accordance with various embodiments described herein. An inner, porous pouch assembly containing a plurality of compressed cellulose sponge articles was first prepared. In one embodiment, the porous pouch was formed from a tubular knit textile constructed from ultra-high molecular weight polyethylene fibers and filled with approximately thirty-eight compressed cellulose sponge articles. Each sponge article had a compressed height of approximately 5 mm and a circular diameter of approximately 9 mm. The sponge articles were positioned within the tubular pouch. A radiopaque filament formed from a barium sulfate-infused polymer was positioned along the interior of the pouch to permit radiographic detection of the dressing during use. Following preparation of the inner pouch, the pouch assembly (pouch and liquid-expandable layer) was positioned between top and bottom layers of nonwoven spunlace material composed of viscose and polyester fibers. In one exemplary embodiment, the top and bottom layers were provided as two approximately 10 cm×10 cm sheets of the spunlace material. The pouch assembly was placed between the top and bottom outer layers such that the outer layers surrounded the inner pouch. The outer layers were then joined together along their periphery using stitching with a polyester thread to form a completed wound dressing assembly having approximate dimensions of 10 cm×10 cm (about 4 in×4 in).

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A wound dressing, comprising:
an outer layer having a first liquid permeability;
an inner pouch positioned within the outer layer, the inner pouch having a second liquid permeability; and
a liquid-expandable layer positioned within the inner pouch, wherein the liquid-expandable layer comprises a plurality of liquid-expandable articles,
wherein the first liquid permeability is lower than the second liquid permeability.

2. The wound dressing of claim 1, wherein the first liquid permeability is 1-25% lower than the second liquid permeability.

3. The wound dressing of claim 1, wherein the plurality of liquid-expandable articles comprises compressed sponge.

4. The wound dressing of claim 3, wherein the compressed sponge comprises regenerated cellulose.

5. The wound dressing of claim 1, wherein the liquid-expandable articles comprise a substantially cylindrical shape.

6. The wound dressing of claim 1, wherein the liquid-expandable articles expand to 80% or greater of their maximum expansion capacity within 30 seconds following immersion in water or saline.

7. The wound dressing of claim 1, wherein a volume ratio of expanded articles to liquid-expandable articles is at least 4:1.

8. The wound dressing of claim 1, wherein a pre-expansion volume of each liquid-expandable article is from 0.7 mm³ to 7000 mm³.

9. The wound dressing of claim 1, further comprising one or more therapeutic agents coupled to at least one of the liquid-expandable articles, the outer layer, and the inner pouch.

10. The wound dressing of claim 1, wherein the one or more therapeutic agents comprises a metal ion.

11. The wound dressing of claim 1, wherein the one or more therapeutic agents comprises copper.

12. The wound dressing of claim 1, wherein the one or more therapeutic agents comprises silver.

13. The wound dressing of claim 1, wherein the one or more therapeutic agents comprises an antimicrobial agent.

14. The wound dressing of claim 1, wherein the one or more therapeutic agents comprises a hemostatic agent.

15. The wound dressing of claim 1, wherein the one or more therapeutic agents comprises a hemostatic agent.
